(19) United States
(12) Patent Application Publication
(10) Pub. No.: US 2024/0408360 A1
(43) Pub. Date: Dec. 12, 2024

(54) OPTICAL DIAGNOSIS OF SHUNT FAILURE IN PEDIATRIC HYDROCEPHALUS

(71) Applicant: THE CHILDREN'S HOSPITAL OF PHILADELPHIA, Philadelphia, PA (US)

(72) Inventors: Wesley BAKER, Philadelphia, PA (US); Daniel J. LICHT, Philadelphia, PA (US); Rodrigo FORTI, Philadelphia, PA (US); Tiffany KO, Philadelphia, PA (US); Todd KILBAUGH, Philadelphia, PA (US)

(73) Assignee: THE CHILDREN'S HOSPITAL OF PHILADELPHIA, Philadelphia, PA (US)

(21) Appl. No.: 18/702,839

(22) PCT Filed: Oct. 20, 2022
(86) PCT No.: PCT/US2022/047215

§ 371 Date: Apr. 19, 2024

Related U.S. Application Data
(60) Provisional application No. 63/257,685, filed on Oct. 20, 2021.

(51) Int. Cl. A61M 27/00

(52) U.S. Cl. CPC A61M 27/006; A61M 2205/3306; A61M 2205/3334; A61M 2205/3344; A61M 2205/50

(57) ABSTRACT

A cerebral monitoring device for determining failure of a shunt used to treat pediatric hydrocephalus. The cerebral monitoring device including a controller configured to control an optical instrument to emit multi-spectral light to illuminate a cranial tissue of the patient, and control an optical detector to detect multi-spectral light emitted from the illuminated cranial tissue of the patient. The controller also configured to compare the emitted multi-spectral light to the detected multi-spectral light, compute cerebral blood flow (CBF) data based on the comparison, compute a pulsatility index of the CBF data, compute a pulsatility index of blood pressure of the patient, compute intracranial pressure (ICP) based on the pulsatility index of the CBF data and the pulsatility index of the blood pressure, and determine shunt failure based on blood oxygen saturation of the patient and the ICP of the patient.

This is the U.S. National Phase application of PCT/US2022/047215, filed Oct. 20, 2022, the disclosure of this application being incorporated herein by reference in its entirety for all purposes which claims priority to U.S. Provisional Application Ser. No. 63/257,685, which is incorporated herein by reference.

### STATEMENT REGARDING FEDERALLY SPONSORED RESEARCH

This invention was made with government support under grant number NS113945 awarded by the National Institutes of Health. The government has certain rights in the invention.

### FIELD OF THE INVENTION

The subject matter disclosed herein relates to devices, systems and methods for providing non-invasive cerebral monitoring diagnosis of a failure of a shunt used in the treatment of pediatric hydrocephalus.

### BACKGROUND OF THE INVENTION

Hydrocephalus is a common disorder of cerebral spinal fluid (CSF) physiology that produces increased intracranial pressure (ICP) on the brain. One neurosurgical treatment for hydrocephalus is ventriculoperitoneal (VP) shunt placement, which diverts CSF from the cerebral ventricles to the abdomen to relieve elevated ICP. Failure of a VP shunt requires revision/replacement and is very common, occurring in approximately 40% of children within the first 2 years after original placement. Current diagnosis of shunt failure relies on imaging evidence of ventricular change and clinical judgement. Ventricular size, however, is a suboptimal predictor for surgical intervention, in part because its relationship with elevated ICP is inconsistent.

### SUMMARY OF THE INVENTION

In one aspect, there is provided a cerebral monitoring device for determining failure of a shunt used to treat pediatric hydrocephalus. The cerebral monitoring device including a measurement probe having one or more optical emitters, and one or more optical detectors, and including an optical instrument having an optical source, and an optical detector. The device also includes a controller configured to control the optical instrument to emit multi-spectral light through the one or more optical emitters to illuminate a cranial tissue of the patient, control the optical detector to detect multi-spectral light emitted from the illuminated cranial tissue of the patient, compare the emitted multi-spectral light to the detected multi-spectral light, compute cerebral blood flow (CBF) data based on the comparison, compute a pulsatility index of the CBF data, compute a pulsatility index of blood pressure of the patient, compute intracranial pressure (ICP) based on the pulsatility index of the CBF data and the pulsatility index of the blood pressure, and determine shunt failure based on blood oxygen saturation of the patient and the ICP of the patient.

In another aspect, there is provided a cerebral monitoring method using a cerebral monitoring device for determining failure of a shunt used to treat pediatric hydrocephalus. The method includes controlling, by a processor of the cerebral monitoring device, an optical instrument placed to emit multi-spectral light through the one or more optical emitters to illuminate a cranial tissue of the patient, controlling, by the processor, an optical detector to detect multi-spectral light emitted from the illuminated cranial tissue of the patient, comparing, by the processor, the emitted multi-spectral light to the detected multi-spectral light, computing, by the processor, cerebral blood flow (CBF) data based on the comparison, computing, by the processor, a pulsatility index of the CBF data, computing, by the processor, a pulsatility index of blood pressure of the patient, computing, by the processor, intracranial pressure (ICP) based on the pulsatility index of the CBF data and the pulsatility index of the blood pressure, and determining, by the processor, shunt failure based on blood oxygen saturation of the patient and the ICP of the patient.

### BRIEF DESCRIPTION OF THE FIGURES

FIG. 1A is a view of a pediatric patient implanted with a shunt for treating pediatric hydrocephalus, according to an aspect of the disclosure.

FIG. 1B is a view of an operational flow for non-invasive cerebral monitoring and diagnosis of a failure of a shunt used in the treatment of pediatric hydrocephalus, according to an aspect of the disclosure.

FIG. 2A is a view of a control system for non-invasive cerebral monitoring and diagnosis of a failure of a shunt used in the treatment of pediatric hydrocephalus, according to an aspect of the disclosure.

FIG. 2B is a detailed view of the control system for non-invasive cerebral monitoring and diagnosis of a failure of a shunt used in the treatment of pediatric hydrocephalus shown in FIG. 2A, according to an aspect of the disclosure.

FIG. 3 is a flowchart of the operational steps to perform the non-invasive cerebral monitoring and diagnosis of a failure of a shunt used in the treatment of pediatric hydrocephalus as in FIG. 1B, according to an aspect of the disclosure.

FIG. 4 is a more detailed flowchart of the operational steps to perform the non-invasive cerebral monitoring and diagnosis of a failure of a shunt used in the treatment of pediatric hydrocephalus as in FIG. 3, according to an aspect of the disclosure.

FIG. 5 is a detailed flowchart of the operational steps to perform the analysis step in in FIG. 4, according to an aspect of the disclosure.

FIG. 6 is a graph of data collected using Optical ICP methods on a population of patients.

### DETAILED DESCRIPTION OF THE INVENTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, components, and circuitry have been described at a relatively high-level, without detail, in order to avoid unnecessarily obscuring aspects of the present teachings.

### Introduction

The following description describes systems and methods for providing non-invasive cerebral monitoring diagnosis of a failure of a shunt used in the treatment of pediatric hydrocephalus.

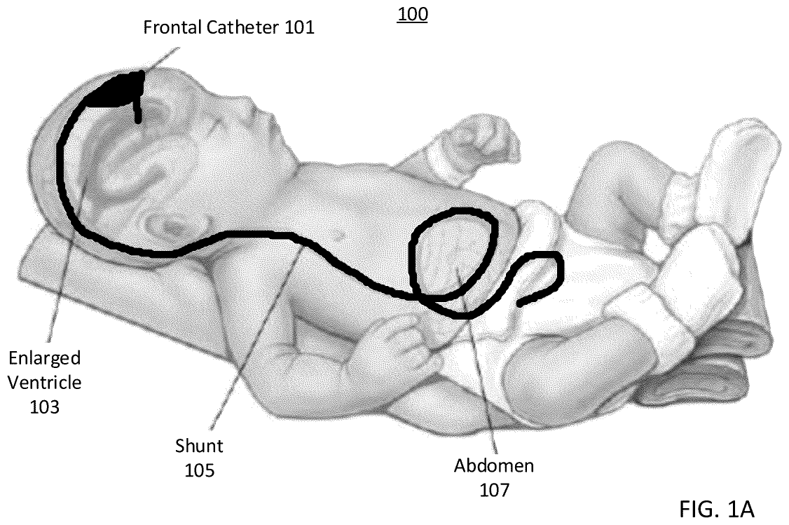

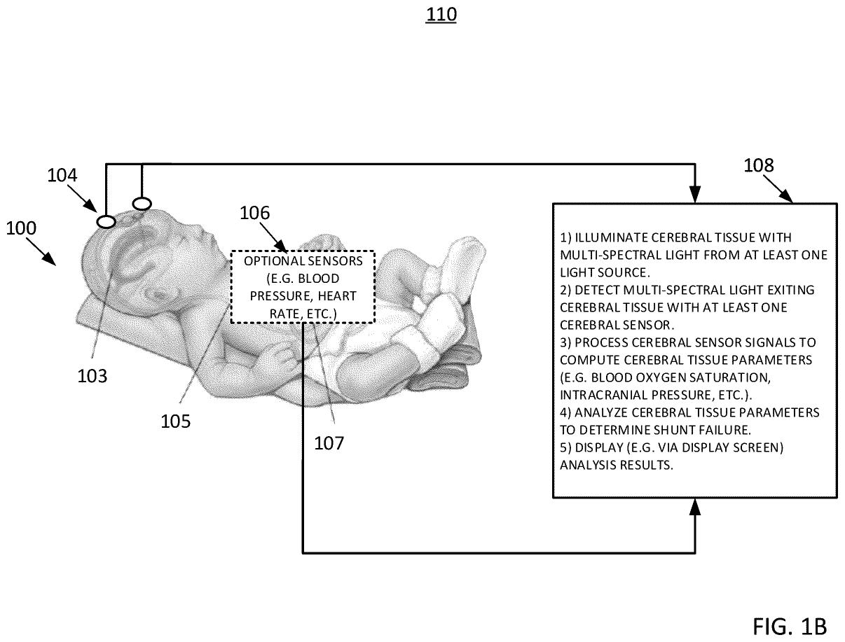

FIG. 1A is an example of pediatric patient 100 suffering from pediatric hydrocephalus. Pediatric hydrocephalus is a condition where excess fluid fills a ventricle in the brain, which causes an enlarged ventricle 103 and a resultant increased intracranial pressure (ICP). Upon diagnosis of pediatric hydrocephalus, ventriculoperitoneal (VP) shunt 105 (herein “shunt”) is surgically implanted into the ventricle along with frontal catheter 101 to allow excess fluid in the ventricle to drain into another part of the body (e.g., abdomen 107 as shown in FIG. 1A). However, over time, shunt failure is known to occur. Failures may be caused for numerous reasons, but generally fall into one of two categories (e.g., shunt becomes blocked with biological matter, and/or shunt loses structural integrity causing it to pinch shut or crack) which once again leads to buildup of excess fluid in the brain and a resultant increased ICP.

FIG. 1B is an example of an operational flow 110 for quickly and accurately diagnosing such shunt failure based on non-invasive cerebral monitoring. In FIG. 1B, for example, one or more optical measurement probes 104, and optional sensors 106 (e.g., blood pressure, peripheral blood flow (including carotid blood flow) and heart rate) may be attached to pediatric patient 100 by a caregiver (not shown). In one example, system 108 includes a controller, user input/output and optional automated medical sensors and devices. System 108 controls optical measurement probes 104 (e.g., attached to the scalp of pediatric patient 100) to illuminate cerebral tissue with light (e.g., multi-spectral light) from at least one light source, and then detect light exiting the cerebral tissue with at least one optical detector. Each optical measurement probe 104 includes of at least one emitter-detector pair. System 108 then analyzes cerebral monitoring signals received from optical measurement probes 104 to compute cerebral tissue parameters (e.g., cerebral blood flow (CBF), intracranial pressure, tissue oxygenation, etc.). System 108 then processes these cerebral tissue parameters to determine shunt failure.

### Device Hardware

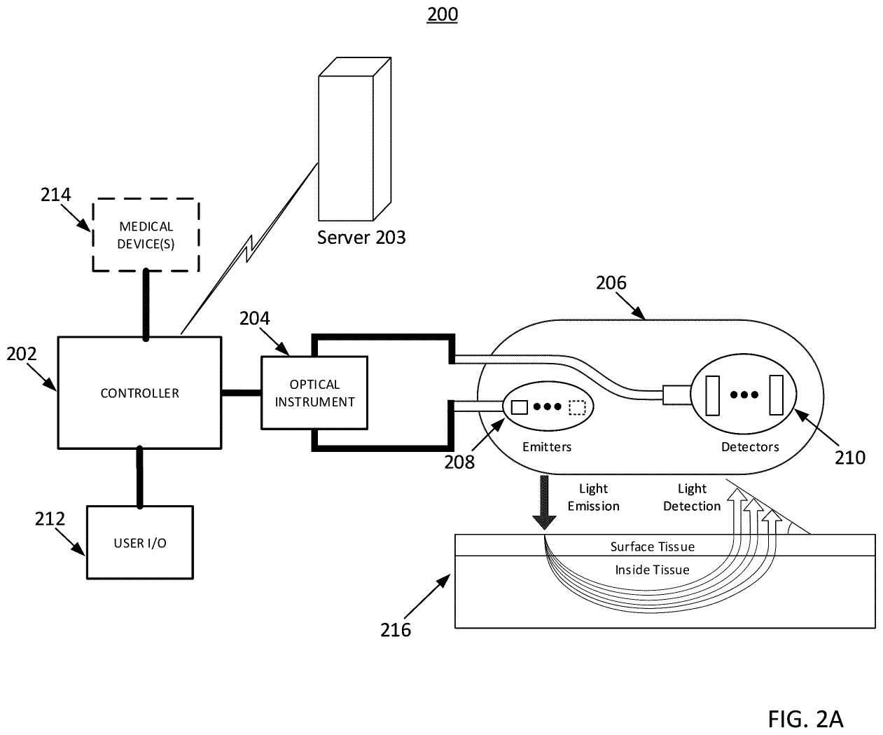

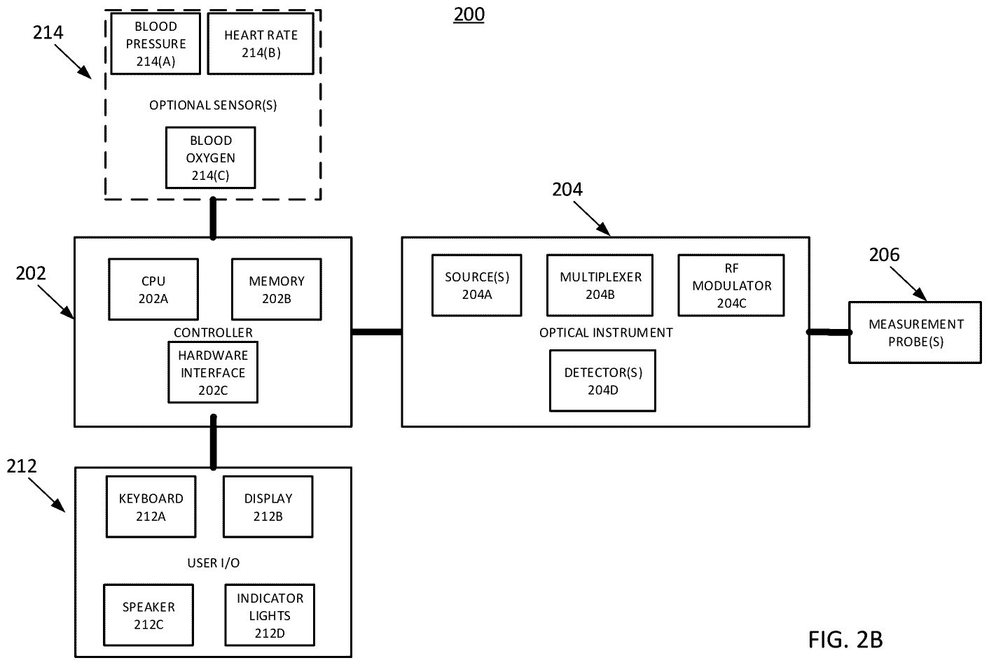

FIG. 2A is a view of a control system 200 for performing the non-invasive cerebral monitoring diagnosis of a failure of a shunt used in the treatment of pediatric hydrocephalus described in FIG. 1B. In this example, control system 200 includes at least one optical measurement probe 206 having one or more optical emitters 208 and one or more optical detectors 210 for illuminating and detecting light, respectively, in tissue 216 (e.g., cerebral tissue), and optical instrument 204 for providing/receiving the light to/from the optical emitters 208 and optical detectors 210. Also included is user (e.g., caregiver) input/output (I/O) device(s) 212, optional medical device(s) 214 (e.g., blood pressure detector, heart rate detector, blood oxygen saturation detector, etc.), controller 202 for controlling the system and optional server 203 for updating software programs and data stored on controller 202.

Further details of example control system 200 are shown in FIG. 2B. In this example, controller 202 includes a central processing unit (CPU) 202A for processing data, memory 202B for storing data and software programs and hardware interface 202C for interfacing CPU 202A to the other hardware devices in the system. Optical instrument 204 includes one or more optical sources 204A (e.g., lasers of different wavelengths or a multispectral light source) for outputting multi-spectral light to measurement probes 206 (e.g., via optical fiber), optical multiplexer 204B for time division multiplexing of optical sources 204A (e.g., multiplexing the lasers of different wavelengths to produce multispectral light), radio frequency (RF) optical modulator 204C for optically modulating the multiplexed optical sources to RF frequencies, and then outputting light to measurement probes 206, and one or more optical detectors 204D (e.g., photodiodes) for detecting the light detected by measurement probes 206.

The power, coherence, number and emission wavelengths of optical sources 204A are set based on various factors including optical measurement technique (e.g., frequency-domain versus time-domain diffuse optical spectroscopy), required measurement time resolution, the anatomical region of measurement, and cerebral tissue parameters that are of importance for detecting shunt failure. In addition, the number and positioning of optical emitters 208 and optical detectors 210 are also set based on these factors. At minimum, the system uses a least one optical source and at least one detector. However, more optical sources and detectors may be utilized to improve accuracy of cerebral parameter detection.

For example, the optical instrument 204 in the system may include eight optical sources 204A (e.g., lasers), comprising two duplicated sets of four unique near-infrared wavelengths (multi-spectral), and the measurement probes 206 may each include two optical emitters 208 spaced at various distances from a single optical detector 210. In operation, CPU 202A controls multiplexer 204B to sequentially output each of the first set of four lasers from the first optical emitter, followed by sequentially outputting each of the second set of the four lasers from the second optical emitter. This produces 8 independent emissions and detections of the laser light through the cerebral tissue which is then analyzed by CPU 202A to determine the cerebral tissue parameters.

In addition, the system also includes user I/O 212 having one or more of keyboard 212A, display 212B, haptic feedback device 212C, speaker 212D, virtual reality device 212E and indicator lights 212F for receiving input (e.g., patient information) and providing output (e.g., diagnosis result of the shunt) to the caregiver. In addition, optional medical devices 214 include one or more of blood pressure detector 214A, heart rate detector 214B and blood oxygen saturation detector 214C. Other medical devices may also be included depending on the health of pediatric patient 100.

### Operational Overview and Signal Processing

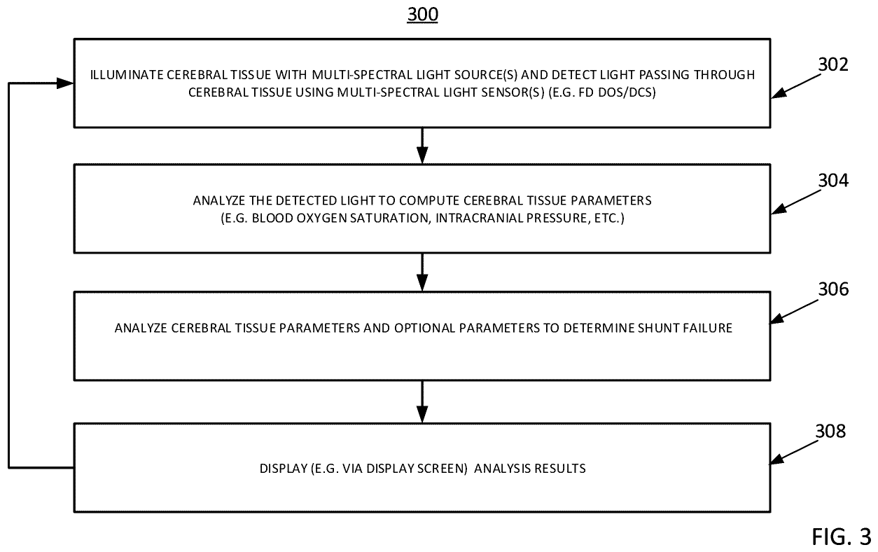

FIG. 3 is a flowchart 300 describing the operational flow for providing non-invasive cerebral monitoring diagnosis of shunt failure as shown in FIG. 1B utilizing control system 200 shown in FIGS. 2A and 2B. In step 302, CPU 202A of controller 202 executes a computer program stored in memory 202B. The computer program instructs CPU 202A to control the optical instrument 204 to illuminate the cerebral tissue of pediatric patient 100 with light (e.g., multi-spectral light) via optical emitter(s) 208, and to detect light passing through the cerebral tissue via optical detector(s) 210. In one example, CPU 202A may control optical multiplexer 204B to perform time division multiplexing to sequentially drive the source lasers' 204A output (each for a period of time T, each with specific emission wavelength), which are amplitude modulated using RF optical modulator 204C onto an RF optical carrier (e.g., 110 MHz), for transmission of light to optical emitter(s) 208, each with a specific position on an optical probe 206. In step 304, the light detected at optical detector positions 210 and transmitted to optical detector(s) 204D is then analyzed by CPU 202A to compute cerebral tissue parameters, which include but are not limited to optical scattering and absorption properties (μₛ′ and μₐ, respectively), blood flow (e.g., CBF), tissue oxygenation, hemoglobin concentration and cerebral metabolism. More specifically, steps 302 and 304 may include a combination of frequency domain diffuse optical spectroscopy (FD-DOS) and diffuse correlation spectroscopy (DCS) referred to herein as FD-DOS/DCS to dictate light emission and detection schema (e.g., optical instrument 204 and measurement probe 206), analyze the light signals and compute the cerebral tissue parameters. FD-DOS uses RF amplitude modulated laser sources to quantify both optical scattering and optical absorption in the tissue which is beneficial to more accurately determine the cerebral tissue parameters, as compared to optical instruments where RF modulation and resulting phase information is not used. DCS uses speckle intensity fluctuations of detected multiply-scattered coherent light to quantify CBF at high temporal resolution (e.g., 20 Hz sampling). More generally, any optical instrument 204 and measurement probe 206 configuration which is able to quantify optical scattering and optical absorption at multiple wavelengths may be used in place of FD-DOS. Similarly, any optical instrument 204 and measurement probe 206 configuration which permits optical measurement of CBF may be used in place of DCS.

Alternatively, steps 302 and 304 may include a combination of time domain diffuse optical spectroscopy (TD-DOS) and DCS. Further details of hybrid diffuse reflectance spectroscopy techniques, FD-DOS/DCS, TD-DOS/DCS, alternative optical instruments and probe configurations can be found in in U.S. Pat. No. 8,082,015 and PCT/US2020/058809, which are both incorporated herein by reference for all purposes.

Based on the cerebral tissue parameters determined in step 304, and possibly based on optional parameters determined from optional sensors 106 and optional patient information input via user I/O 212 and CPU 202A, in step 306 CPU 202A then analyzes the parameters to determine shunt failure. CPU 202A makes this determination by computing pulsatility indexes for one or more of the parameters to determine ICP and then comparing the ICP, and parameters, to respective thresholds that are correlated with shunt failure. CPU 202A may perform this analysis as a standalone device, or in conjunction with server 203.

In step 308, CPU 202A then indicates shunt status to the caregiver (e.g., medical professional) via a display 212B, speaker 212C or indicator lights 212D. For example, display 212B may display text indicating whether the shunt has failed, or is still operational. This indication may be a hard indication (e.g., “Fail” or “Pass”), or a soft indication (e.g., “90% probability that shunt has failed”). Furthermore, the indication may state the severity of the failure (e.g., “Shunt is operating at 50% capacity due to apparent blockage”), or even a prediction of failure (e.g., “Shunt will likely fail within 3 months”).

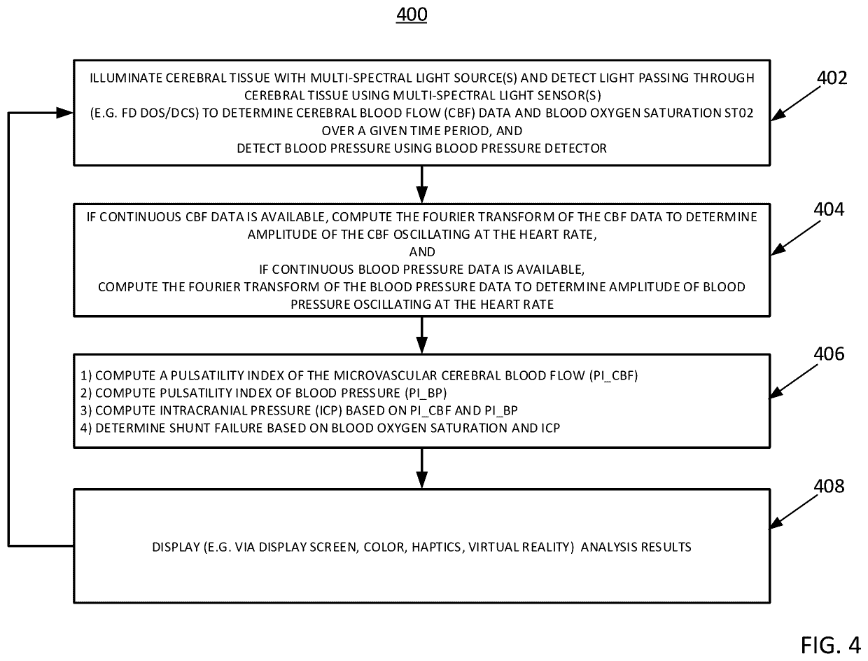

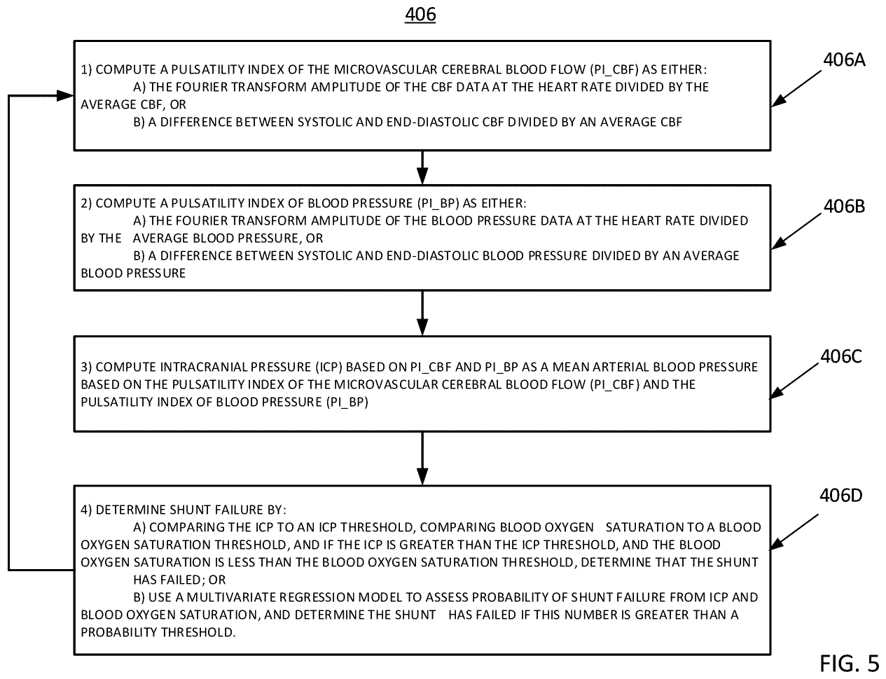

The overall operational flow of control system 200 is described above with respect to FIG. 3. However, a specific implementation will now be described with respect to FIGS. 4 and 5.

FIG. 4 is a detailed implementation example of the operational flow shown in FIG. 3. It will be understood that FIG. 4 is just one implementation of the operational flow. Other implementations of the operational flow are possible. FIG. 5 is a detailed example of the analysis step shown in FIG. 4. It will be understood that FIG. 5 is just one implementation of the analysis step. Other implementations of the analysis step are possible.

In step 402, CPU 202A of controller 202 executes a computer program stored in memory 202B. The computer program instructs CPU 202A to control the optical instrument 204 to illuminate the cerebral tissue of pediatric patient 100 with light (e.g., multi-spectral light) via optical emitter(s) 208, and to detect light passing through the cerebral tissue via optical detector(s) 210 that are placed on the cerebral tissue in proximity to affected brain ventricle being drained by the shunt. The light detected at optical detector positions 210 and transmitted to optical detector(s) 204D is then analyzed at a specified rate (e.g., at a 20 Hz sampling rate) by CPU 202A for a specified period of time (e.g., ˜1-2 minutes) to compute cerebral tissue parameters including microvascular oxygen saturation (StO2) and cerebral blood flow (CBF) measurements. In this example, CPU 202A uses frequency domain diffuse optical spectroscopy (FD-DOS) to compute StO2, and uses diffuse correlation spectroscopy (DCS) to compute CBF. In addition, blood pressure of pediatric patient 100 is measured either continuously, or through a cuff.

If continuous CBF data is available, CPU 202A then, in step 404, computes a frequency domain transform of the CBF data in step 404 to determine an amplitude of the CBF as it oscillates with the period of the heart rate. If continuous blood pressure data is available, CPU 202A also computes a frequency domain transform of the blood pressure data in step 404 to determine an amplitude of the blood pressure data as it oscillates with the period of the heart rate. The frequency domain transform may be a Fast Fourier Transform (FFT) or some other equivalent transformation.

In step 406, CPU 202A then analyzes the CBF data to determine indices indicative of ICP, which is then used along with blood oxygen saturation to determine shunt failure. As shown in step 406A in FIG. 5, if continuous CBF data is available, the Fourier amplitude of the CBF data determined in step 404 is divided by the mean CBF across the 1-2 minutes of CBF data to compute a microvascular CBF waveform pulsatility index (PI_CBF). If continuous CBF data is not available, PI_CBF is computed as a difference between systolic and end-diastolic CBF divided by an average CBF (e.g., average CBF over a time period).

Likewise, CPU 202A also computes an arterial blood pressure waveform pulsatility index (PI_BP). As shown in step 406B in FIG. 5, if continuous arterial blood pressure data is available over the 1-2 minutes, PI_BP is computed in a manner similar to PI_CBF (e.g., the Fourier amplitude of blood pressure data determined in step 404 is divided by an average blood pressure across the 1-2 minutes of blood pressure data). If continuous blood pressure data is not available (e.g., only discrete cuff blood pressure data is available), PI_BP is computed as a difference between systolic and end-diastolic blood pressure divided by an average blood pressure (e.g., average blood pressure over a time period).

CPU 202A then computes ICP based on these indices. For example, ICP can be computed (see step 406C in FIG. 5) as: ICP=0.6*MAP*(1−PI_ABP/PI_CBF), where MAP is the mean arterial blood pressure. It is noted that ICP as described above is strongly correlated with ICP that is measured invasively, and therefore is an accurate representation of intracranial pressure.

Once ICP is computed, CPU 202A then determines if the shunt has failed. This determination is made by CPU 202A by either threshold comparison or by multivariate regression modeling.

In a first thresholding example, shunt failure is determined by comparing ICP to an ICP threshold, and comparing StO2 to a StO2 threshold (see step 406D in FIG. 5). For example, if StO2<55% (e.g., blood oxygen levels are low), and ICP>20 mmHg (intracranial pressure is high), CPU 202A would determine that the shunt has failed. In contrast, if StO2≥55% (e.g., blood oxygen levels are not low), or ICP≤20 mmHg (intracranial pressure is not high), CPU 202A would determine that the shunt has passed (i.e. shunt is still operational).

In a second thresholding example, StO2 and ICP data may be compared to multiple StO2 thresholds and/or multiple ICP thresholds, by CPU 202A to indicate probability (e.g., soft decision) of shunt failure. In this example, probability of shunt failure may be a product of where StO2 and ICP stand relative to the multiple thresholds and based on clinical data of other patients with failed shunts. For example, if there are three ICP thresholds and three StO2 thresholds, the CPU may indicate: a) a low severity failure when the StO2 is less than one of the three StO2 thresholds and ICP is greater than one of the three ICP thresholds, b) a medium severity failure when the StO2 is less than two of the three StO2 thresholds and ICP is greater than two of the three ICP thresholds, and c) a high severity failure when the StO2 is less than all three StO2 thresholds and ICP is greater than all three ICP thresholds.

In a modeling example, a multivariate logistic regression model of StO2 and ICP data may be used in combination to indicate probability (e.g., soft decision) of shunt failure. As in the univariate models, a ROC analysis may be used to determine the optimal threshold from the multivariate model that best predicts shunt failure. For example, data collected from patients that are measured with functioning shunts at one visit, and return at a future visit with a failed shunt, may be used to develop predictive models of a future shunt failure (e.g., “Shunt is currently operational but will likely fail within 3 months”). This may be performed by applying convolutional and recurrent neural network techniques, as well as multivariate logistic regression, to time-varying features in the StO2 and ICP measurements. These features are in addition to the average StO2 and ICP levels. Such time-varying features may include low-frequency (0.01 Hz to 0.1 Hz) spectral power of the StO2 and CBF signals.

To identify optimal ICP and StO2 thresholds to diagnose shunt failure, a logistic regression may be used to analyze clinical data of a sample of pediatric patients with shunts. Specifically, the optical ICP and StO2 measurements may be dichotomized into failed shunt and functioning shunt groups. A logistic regression model, followed by a receiver operating characteristic (ROC) analysis, may then be used to determine optimal ICP and StO2 thresholds that best predict shunt failure based on maximal sum of true positive and false negative rates. Doing this analysis on a pilot cohort of 21 hydrocephalus children (10 of whom had shunt failure), Applicant identified ICP and StO2 thresholds of 20 mmHg and 55%, respectively. Other methods are of course possible for determining optimal threshold values.

In another example, and adaptive filter that learns from the success (or lack of success) of prior attempts to predict shunt failure may be used to determine and/or refine optimal thresholds for ICP and StO2. Such an algorithm can be initialized based on clinical data and optimized over time. This would also allow for the system to adapt and be optimized to a particular patient over multiple sessions.

In step 308, CPU 202A then indicates shunt status to the caregiver (e.g., medical professional) via a display 212B, speaker 212C or indicator lights 212D. For example, display 212B may display text indicating whether the shunt has failed, or is still operational. This indication may be a hard indication (e.g., “Fail” or “Pass”).

It is noted that the processes described herein can be performed in response to symptomatic behavior of the pediatric patient, or as a routine to periodically monitor the integrity of the shunt. In such an example, periodic measurements may be compared to determine if the StO2 and ICP levels are trending in a direction over time where they are approaching the failure thresholds. Such a trend could indicate that the shunt is beginning to fail (e.g., gradually becoming obstructed due to biological material).

It is also noted that although this disclosure focuses on diagnosis of shunt failure of a pediatric patient, the method/systems described herein are also applicable for diagnosis of shunt failure in an adult patient.

Furthermore, it is noted that although it is described above that CPU 202A performs all the method steps, it is contemplated that server 203 may perform the method steps, or work in conjunction with CPU 202A to perform the method steps.

The steps in FIGS. 1 and 3-5 may be performed by the controller 202 in FIGS. 2A and 2B, upon loading and executing software code or instructions which are tangibly stored on a tangible computer readable medium, such as on a magnetic medium, e.g., a computer hard drive, an optical medium, e.g., an optical disc, solid-state memory, e.g., flash memory, or other storage media known in the art. In one example, data are encrypted when written to memory, which is beneficial for use in any setting where privacy concerns such as protected health information is concerned. Any of the functionality performed by the computer (e.g., controller having a processor, server, etc.) described herein, such as the steps in FIGS. 1 and 3-5 may be implemented in software code or instructions which are tangibly stored on a tangible computer readable medium. Upon loading and executing such software code or instructions by the computer (e.g., controller having a processor, server, etc.), the computer (e.g., controller having a processor, server, etc.) may perform any of the functionality of the computer described herein, including the steps in FIGS. 1 and 3-5 described herein.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that comprises or includes a list of elements or steps does not include only those elements or steps but may include other elements or steps not expressly listed or inherent to such process, method, article, or apparatus. An element preceded by “a” or “an” does not, without further constraints, preclude the existence of additional identical elements in the process, method, article, or apparatus that comprises the element.

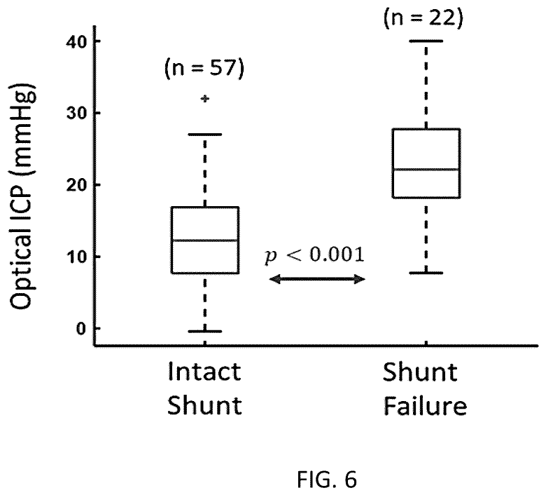

FIG. 6 illustrates the results of a study performed by the Applicant. In this case, the study was performed on children who presented in an emergency room with symptoms of shunt failure. The children were optically measured prior to a clinical diagnosis of whether the shunt was broken or not. An optical intracranial pressure (ICP) measurement was made using methods described herein, and it was determined that the ICP measurement was higher in children with actual shunt failure (n=22) than it was in children with intact shunts (n=57). The boxplots in FIG. 6 show the median and interquartile range for the data of each population. As indicated by the calculated probability value (i.e., p<0.001) the data has statistical significance, leading to the conclusion that optical ICP can be used to diagnose shunt failure prior to the clinical diagnosis. This earlier diagnosis enables earlier treatment that may save brain tissue (e.g., in two of the twenty-two children with shunt failure, the clinical diagnosis wasn't made until 1 to 2 weeks after their initial ER visit).

Unless otherwise stated, any and all measurements, values, ratings, positions, magnitudes, sizes, and other specifications that are set forth in this specification, including in the claims that follow, are approximate, not exact. Such amounts are intended to have a reasonable range that is consistent with the functions to which they relate and with what is customary in the art to which they pertain. For example, unless expressly stated otherwise, a parameter value or the like may vary by as much as ±10% from the stated amount.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

While the foregoing has described specific examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present concepts.

1. A cerebral monitoring device for determining failure of a shunt used to treat pediatric hydrocephalus, the cerebral monitoring device comprising:
a measurement probe including:
one or more optical emitters, and one or more optical detectors;
an optical instrument including:
an optical source, and an optical detector; and
a controller configured to:
control the optical instrument to emit multi-spectral light through the one or more optical emitters to illuminate a cranial tissue of the patient,
control the optical detector to detect multi-spectral light emitted from the illuminated cranial tissue of the patient,
compare the emitted multi-spectral light to the detected multi-spectral light,
compute cerebral blood flow (CBF) data based on the comparison,
compute a pulsatility index of the CBF data,
compute a pulsatility index of blood pressure of the patient,
compute intracranial pressure (ICP) based on the pulsatility index of the CBF data and the pulsatility index of the blood pressure, and
determine shunt failure based on blood oxygen saturation of the patient and the ICP of the patient.

2. The cerebral monitoring device of claim 1, wherein the optical instrument and the measurement probe are positioned on the cranium in proximity to the shunt.

3. The cerebral monitoring device of claim 1, wherein the controller is further configured to compute the CBF data as blood volume flowing over a time period.

4. The cerebral monitoring device of claim 1, wherein the controller is further configured to compute:
the pulsatility index of the CBF data as either:
a frequency domain amplitude of the CBF data at a heart rate of the patient divided by an average CBF over a time period, or
a difference between systolic and end-diastolic CBF divided by an average CBF; and
the pulsatility index of the blood pressure as either:
a frequency domain amplitude of the blood pressure at the heart rate of the patient divided by an average blood pressure over the time period, or
a difference between systolic and end-diastolic blood pressure divided by the average blood pressure.

5. The cerebral monitoring device of claim 1, wherein the controller is further configured to compute ICP by computing a mean arterial blood pressure based on the pulsatility index of the CBF data and the pulsatility index of the blood pressure.

6. The cerebral monitoring device of claim 1, wherein the controller is further configured to:
compare the blood oxygen saturation to a blood oxygen saturation threshold, compare the ICP to an ICP threshold, and
determine that the shunt has failed when the blood oxygen saturation is less than the blood oxygen saturation threshold, and the ICP is greater than the ICP threshold.

7. The cerebral monitoring device of claim 6, wherein blood oxygen saturation threshold and the ICP threshold are set based on clinical data including blood oxygen saturation levels and an ICP levels of multiple patients having shunts.

8. The cerebral monitoring device of claim 1, wherein the controller is further configured to:
compare the blood oxygen saturation to a plurality of blood oxygen saturation thresholds, compare the ICP to a plurality of ICP thresholds, and
determine severity of shunt failure based which of the plurality of blood oxygen saturation thresholds that are exceed by the blood oxygen saturation, and based on which of the plurality of ICP thresholds that are exceed by the ICP.

9. The cerebral monitoring device of claim 1, wherein the controller is further configured to compute the blood oxygen saturation and the CBF data by performing frequency-domain diffuse optical spectroscopy (FD-DOS) and diffuse correlation spectroscopy (DCS) techniques using the multi-spectral light.

10. The cerebral monitoring device of claim 1, wherein the shunt failure occurs due to either a blockage due to blood material stuck in the shunt restricting blood flow, or a structural failure that closes the shunt restricting blood flow.

11. A cerebral monitoring method using a cerebral monitoring device for determining failure of a shunt used to treat pediatric hydrocephalus, the method comprising:
controlling, by a processor of the cerebral monitoring device, an optical instrument placed on to emit multi-spectral light through the one or more optical emitters to illuminate a cranial tissue of the patient; controlling, by the processor, an optical detector to detect multi-spectral light emitted from the illuminated cranial tissue of the patient;
comparing, by the processor, the emitted multi-spectral light to the detected multi-spectral light;
computing, by the processor, cerebral blood flow (CBF) data based on the comparison;
computing, by the processor, a pulsatility index of the CBF data;
computing, by the processor, a pulsatility index of blood pressure of the patient;
computing, by the processor, intracranial pressure (ICP) based on the pulsatility index of the CBF data and the pulsatility index of the blood pressure; and
determining, by the processor, shunt failure based on blood oxygen saturation of the patient and the ICP of the patient.

12. The cerebral monitoring method of claim 11, further comprising positioning the optical instrument and the measurement probe on the cranium in proximity to the shunt.

13. The cerebral monitoring method of claim 11, further comprising computing, by the processor, the CBF data as blood volume flowing over a time period.

14. The cerebral monitoring method of claim 11, further comprising:
computing, by the processor, the pulsatility index of the CBF data as either:
a frequency domain amplitude of the CBF data at a heart rate of the patient divided by an average CBF over a time period, or
a difference between systolic and end-diastolic CBF divided by an average CBF; and
computing, by the processor, the pulsatility index of the blood pressure as either:
a frequency domain amplitude of the blood pressure at the heart rate of the patient divided by an average blood pressure over the time period, or
a difference between systolic and end-diastolic blood pressure divided by the average blood pressure.

15. The cerebral monitoring method of claim 11, further comprising computing, by the processor, ICP by computing a mean arterial blood pressure based on the pulsatility index of the CBF data and the pulsatility index of the blood pressure.

16. The cerebral monitoring method of claim 11, further comprising:
comparing, by the processor, the blood oxygen saturation to a blood oxygen saturation threshold, compare the ICP to an ICP threshold; and
determining, by the processor, that the shunt has failed when the blood oxygen saturation is less than the blood oxygen saturation threshold, and the ICP is greater than the ICP threshold.

17. The cerebral monitoring method of claim 16, further comprising setting, by the processor, the blood oxygen saturation threshold and the ICP threshold based on clinical data including blood oxygen saturation levels and an ICP levels of multiple patients having shunts.

18. The cerebral monitoring method of claim 11, further comprising:
comparing, by the processor, the blood oxygen saturation to a plurality of blood oxygen saturation thresholds, compare the ICP to a plurality of ICP thresholds; and
determining, by the processor, severity of shunt failure based which of the plurality of blood oxygen saturation thresholds that are exceed by the blood oxygen saturation, and based on which of the plurality of ICP thresholds that are exceed by the ICP.

19. The cerebral monitoring method of claim 11, further comprising computing, by the processor, the blood oxygen saturation and the CBF data by performing frequency-domain diffuse optical spectroscopy (FD-DOS) and diffuse correlation spectroscopy (DCS) techniques using the multi-spectral light.

20. The cerebral monitoring method of claim 11, wherein the shunt failure comprises a blockage due to blood material stuck in the shunt restricting blood flow, or a structural failure that closes the shunt restricting blood flow.
